(19) United States
(12) Patent Application Publication
(10) Pub. No.: US 2005/0268910 A1
(43) Pub. Date: Dec. 8, 2005

(54) Humidifier bottle

(76) Inventors: Darren D. Nord, Murray, UT (US); Jeffrey Allen Stephens, Boston, MA (US); Joseph Dieudonne Morin, Laguna Niguel, CA (US); Alexander Robin Marsaw JR., Covington, GA (US); Ian Ryan Hartman, Euless, TX (US); Matthew Carl Goit, Titusville, FL (US); John Kimball Barnhill, Sugar City, ID (US); Jason Lynn Ellsworth, Salt Lake City, UT (US)

Correspondence Address: STOEL RIVES LLP - SLC, 201 SOUTH MAIN STREET, ONE UTAH CENTER, SALT LAKE CITY, UT, 84111, US

(21) Appl. No.: 11/094,969

(22) Filed: Mar. 31, 2005

Related U.S. Application Data
(60) Provisional application No. 60/558,194, filed on Mar. 31, 2004.

(51) Int. Cl. A61M 16/16

(52) U.S. Cl. 128/204.14

(57) ABSTRACT

Disclosed are embodiments of a humidifier bottle apparatus. One embodiment of the apparatus includes a main housing, a fill shaft extending at an angle from the main housing, and a substantially dome-shaped main housing cap, which is releasably connected with the main housing. Gas inlet and gas outlet ports are also connected with the main housing cap. A purge valve feature may be included with some embodiments of the humidifier bottle apparatus. The purge valve feature may provide for audible and/or visual warnings of pressure increases within the apparatus.

### RELATED APPLICATIONS

This application claims the benefit under 35 U.S.C. § 119(e) of U.S. Provisional Patent Application Ser. No. 60/558,194, filed Mar. 31, 2004, and titled “Humidifier Bottle,” which is incorporated herein by specific reference.

### TECHNICAL FIELD

The present invention relates generally to the field of medical devices. More specifically, the present invention relates to humidifier bottles for use in moisturizing oxygen and other gases for administration to a patient.

### BRIEF DESCRIPTION OF THE DRAWINGS

Understanding that drawings depict only typical embodiments of the invention and are not therefore to be considered to be limiting of its scope, the invention will be described and explained with additional specificity and detail through the use of the accompanying drawings in which:

FIG. 1 is a perspective view of an embodiment of a humidifier bottle apparatus.

FIG. 2 is a side elevation view of the embodiment shown in FIG. 1.

FIG. 3 is a top plan view of the embodiment shown in FIG. 1.

FIG. 4 is a cross-sectional view taken along line 44 in FIG. 3.

FIG. 5 is a side elevation view of a main housing and fill shaft of a humidifier bottle apparatus.

FIG. 6 is a side elevation view of a main housing and fill shaft like that of FIG. 5 but rotated ninety degrees.

FIG. 7 is a perspective view of another embodiment of a humidifier bottle apparatus.

FIG. 8 is a side elevation view of the embodiment shown in FIG. 8.

FIG. 9 is a side elevation view of a nozzle for use in a gas outlet of a humidifier bottle apparatus.

FIG. 10A is a cross-sectional view of a main housing cap, showing a purge valve with a purge valve body in a resting position within a purge valve housing.

FIG. 10B is a cross-sectional view like that of FIG. 10A with the purge valve body in a raised position above the purge valve housing.

### DETAILED DESCRIPTION OF PREFERRED EMBODIMENTS

Described below are embodiments of humidifier bottles for use in providing moisturized oxygen and other medicinal gases to patients. In the following description, numerous specific details are provided for a thorough understanding of the embodiments of the invention. However, those skilled in the art will recognize that the invention can be practiced without one or more of the specific details, or with other methods, components, materials, etc.

In addition, in some cases, well-known structures, materials, or operations are not shown or described in detail in order to avoid obscuring aspects of the invention. Furthermore, the described features, structures, or characteristics may be combined in any suitable manner in one or more embodiments.

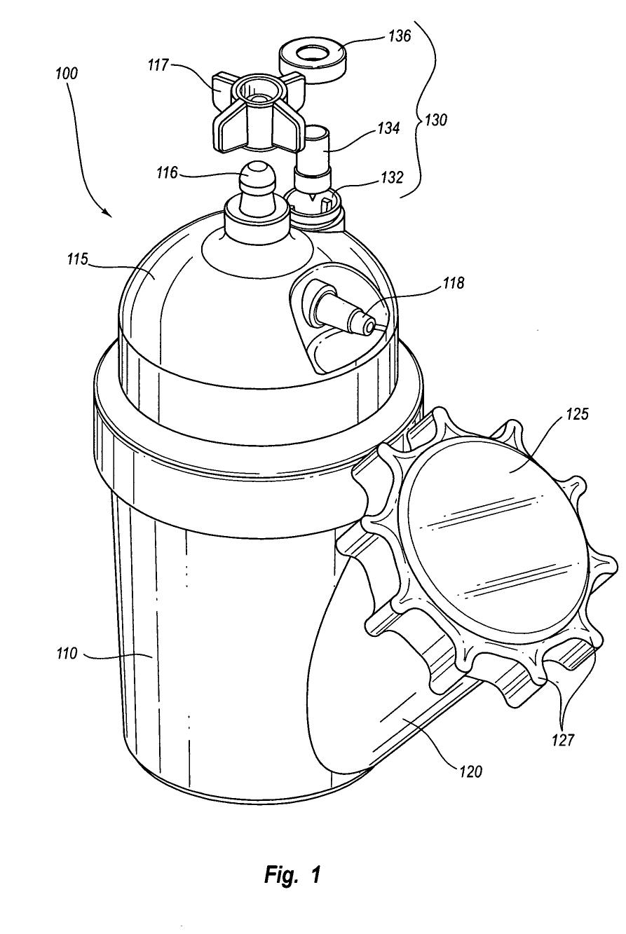

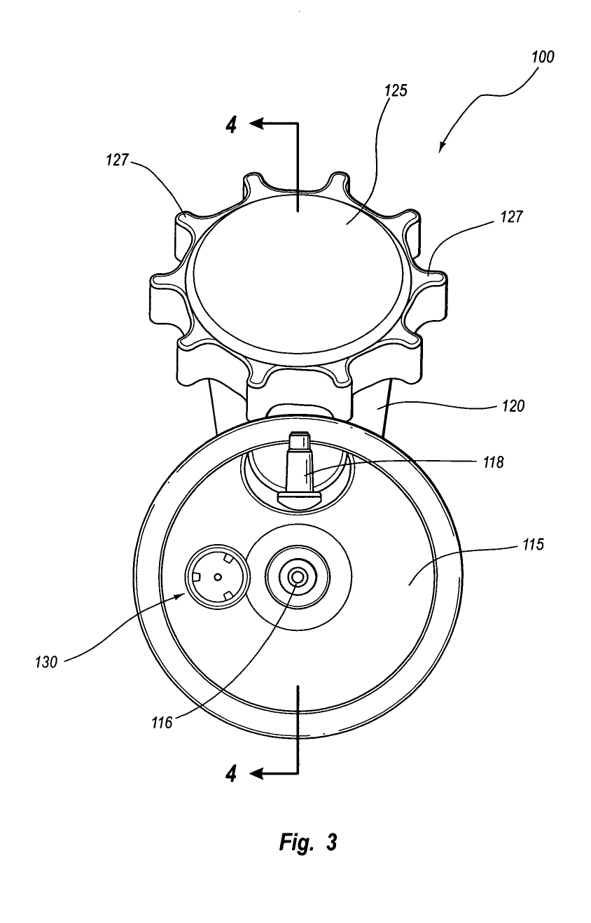

With reference now to FIGS. 1-4, a first embodiment of a humidifier bottle 100 is depicted. Humidifier bottle 100 includes a main housing 110, a main housing cap 115 releasably connected with main housing 110, and a fill shaft 120, which extends at an angle from main housing 110. As can be seen from the figures, main housing cap 115 is substantially dome-shaped, which provides for an increased surface area inside the bottle, thereby providing condensation characteristics that may be desirable for some applications.

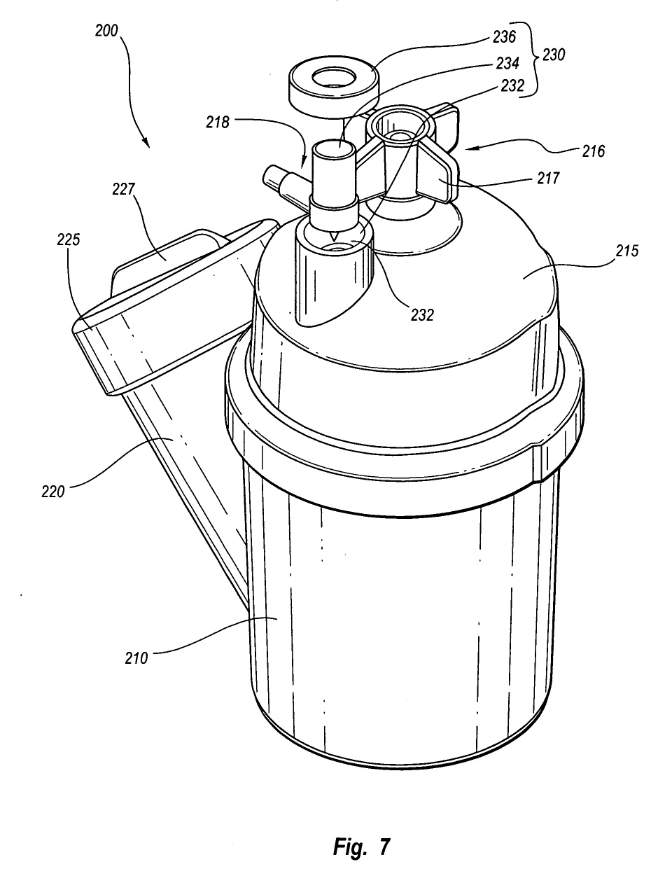

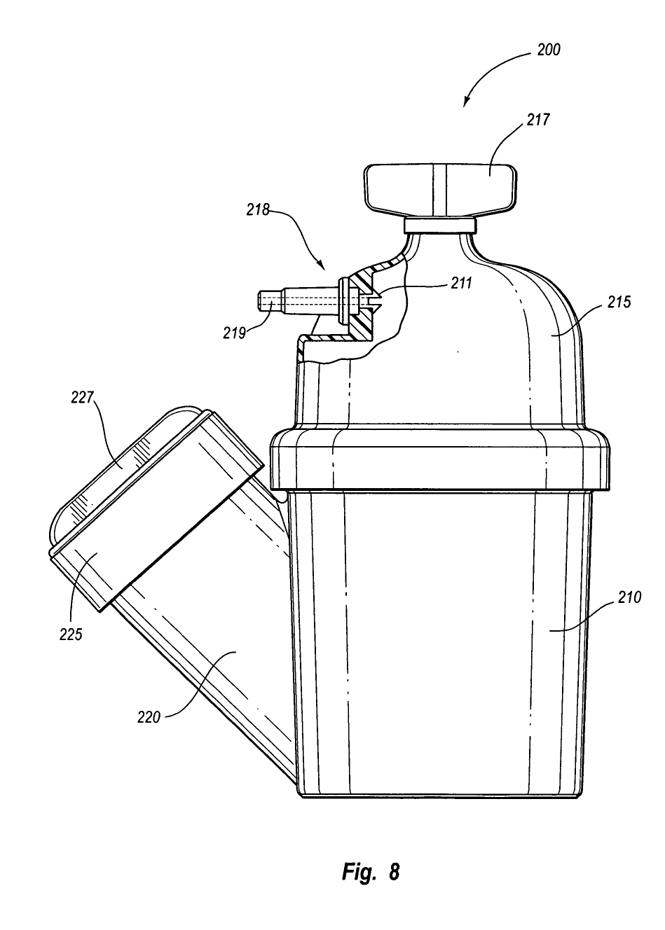

The main housing cap 115 may include a gas inlet 116 and a gas outlet 118. Gas inlet 116 may optionally be connected with an inlet connector piece 117, as shown in FIG. 1, which may be used to facilitate connection of the gas inlet 116 with an oxygen concentrator (not shown) or other supply of medicinal gas. Gas inlet 116 and gas outlet 118 are each integrally formed with the humidifier bottle 100. However, as discussed in greater detail below, either or both of these structures may instead be connected with the bottle. For instance, in another embodiment discussed below with reference to FIGS. 7 and 8, the gas outlet nozzle is configured to rotate or swivel within the gas outlet port.

Optionally, the fill shaft 120 may include a fill shaft cap 125. Fill shaft cap 125 includes a plurality of flanges 127 which may be provided to facilitate removal of the fill shaft cap 125 in order to refill the bottle with water or another liquid.

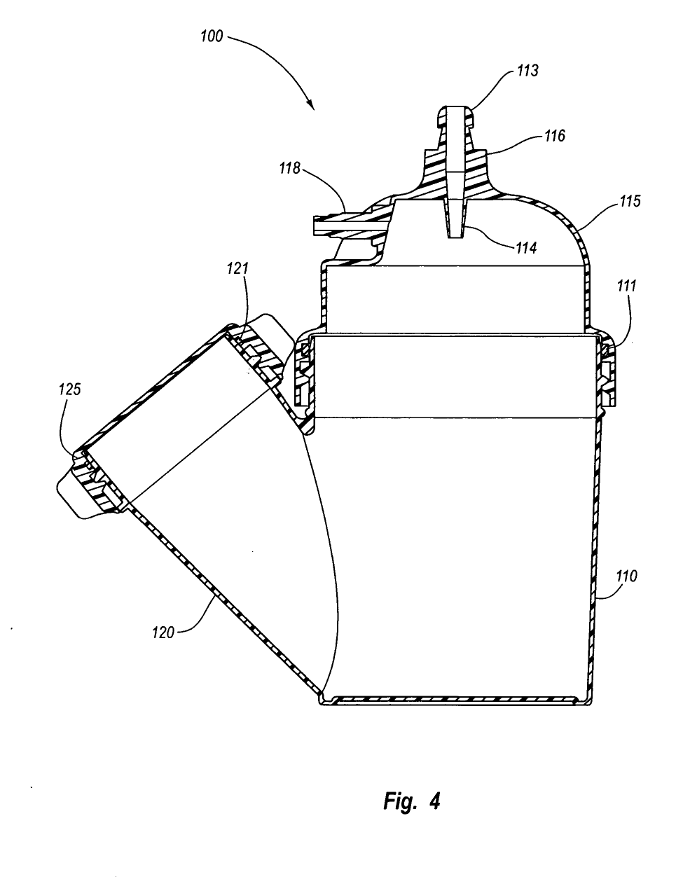

One or more O-rings may also be used to provide a tighter seal between the main housing and the main housing cap and/or between the fill shaft and the fill shaft cap. For instance, in FIG. 4 O-ring 111 is provided in main housing cap 115 and O-ring 121 is provided in fill shaft cap 125.

Humidifier bottle 100 also includes a purge valve 130 configured to provide an audible warning of increased pressure within the humidifier bottle 100. Purge valve 130 includes purge valve housing 132, purge valve body 134, and purge valve cap 136. Purge valve 130 acts not only as an audible gas pressure warning mechanism, but as a visible gas pressure warning mechanism as well, as will be described in greater detail infra.

As previously mentioned, in some embodiments the main housing cap 115 may be configured to have a very large internal surface area to allow for increased condensation and thereby reduce the chances for water being transferred out through the gas outlet 118. The increased surface area may be accomplished by the shape and size of the domed portion of the cap 115, by features protruding from or otherwise formed in the interior surface of the main housing cap 115, or by a combination of the foregoing. Either or both of the main housing cap 115 and fill shaft cap 125 may be configured to be threadably connected with the bottle and may further be provided with suitable threading/groves to prevent cross-threading, as are known and available to one having ordinary skill in the art. For example, a single helical thread may be provided.

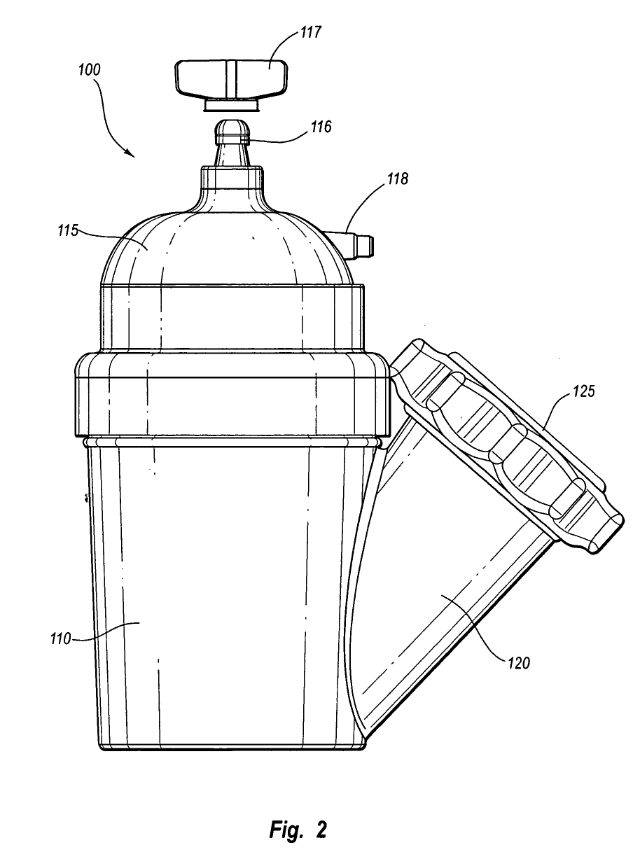

As best seen in FIG. 4, gas inlet 116 includes a nipple portion 113, which may be adapted for attachment to a connector piece, such as connector piece 117 in FIGS. 1 and 2, which may be used to facilitate connection of the gas inlet 116 with an oxygen concentrator or the like. At the end opposite from nipple portion 113, a tubular projection 114 extends into the humidifier bottle 100. Tubular projection 114 is adapted to be connected with a piece of tubing (not shown) extending down into the portion of humidifier bottle 100 defined by the main housing 110.

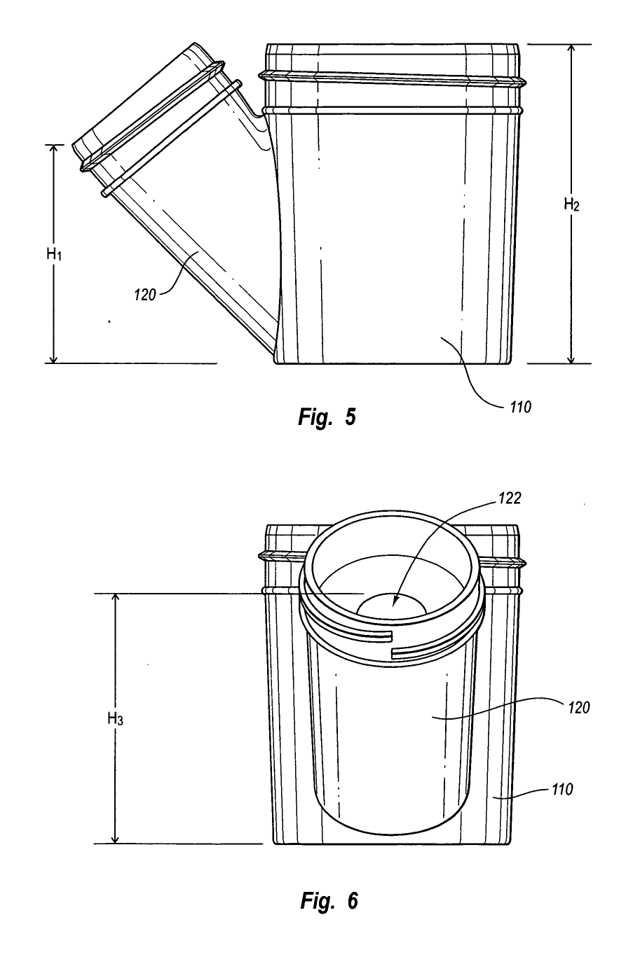

Fill shaft 120 extends at an angle from main housing 110. This allows a patient or caregiver to fill the humidifier bottle without removing the main housing cap 115 and without having to remove or otherwise be concerned with the tubing (not shown) extending from the gas outlet 118. In some embodiments of the invention, the height of the lowest position of the opening in the fill shaft 120 (depicted as height H₁ in FIG. 5) is approximately equal to or less than the height of the main housing 110 (depicted as height H₂ in FIG. 5). This ensures that the main housing 110 cannot be overfilled—i.e., filled above the main housing 110 and into the main housing cap 115. Certain embodiments of the invention may also be configured such that the height of the lowest position of the opening in the fill shaft 120—again depicted as height H₁ in FIG. 5—is approximately equal to the distance from the bottom of the main housing 110 to the top of the opening 122 connecting fill shaft 120 with main housing 110. This distance is depicted as height H₃ in FIG. 6.

A second embodiment of the invention will now be discussed with reference to FIGS. 7 and 8. Humidifier bottle 200 includes a main housing 210, which is adapted to be connected with main housing cap 215. Fill shaft 220 extends at an angle from main housing 210 and is adapted to be connected with fill shaft cap 225. Fill shaft cap 225 includes grip member 227, which allows a user to unscrew fill shaft cap 225 in order to refill the bottle. Main housing cap 215 includes a gas inlet 216 and a gas outlet 218. Gas inlet 216 is shown connected with an inlet connector piece 217.

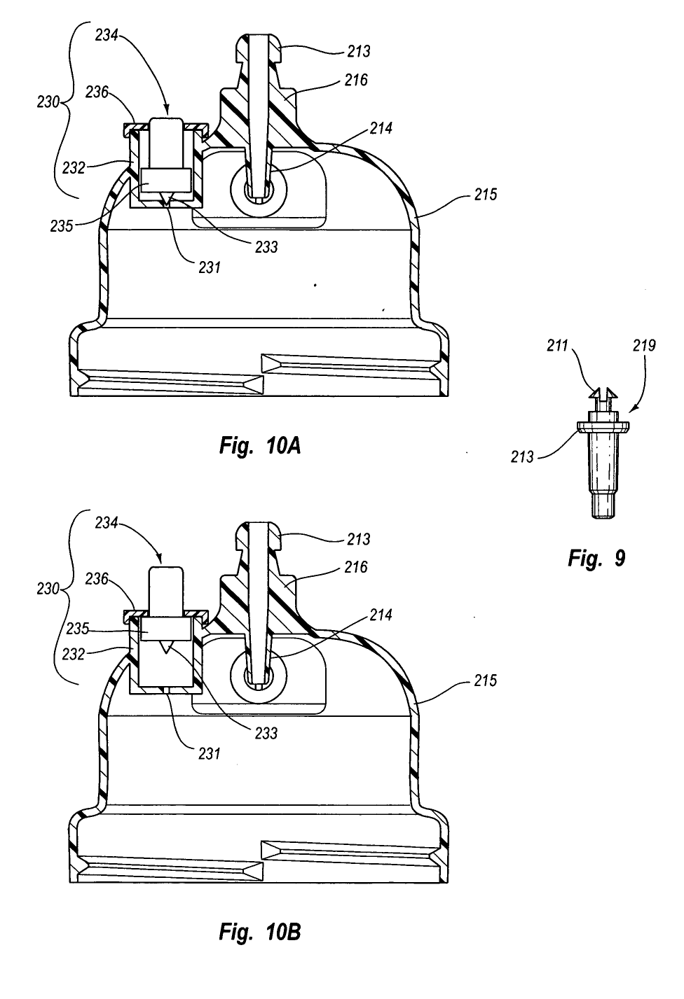

As shown in FIG. 8, gas outlet 218 is configured with a nozzle 219 that is configured to swivel around a horizontal axis within gas outlet 218. This is provided so as to help prevent the associated tubing from becoming kinked. As shown in FIGS. 8 and 9, the nozzle 219 has a pronged portion 211, which fits and allows for rotation within a gas outlet opening in the main housing cap 215. Pronged portion 211 flexes to fit within the gas outlet opening and then engages with the interior surface of main housing cap 215 surrounding the gas outlet opening. Nozzle 219 also includes a widened portion 213, which engages with exterior features on the main housing cap 215. The end of nozzle 219 opposite from pronged portion 211 is configured to allow for tubing to be attached thereto.

With reference now to FIGS. 10A and 10B, a purge valve as provided in some embodiments of the invention will now be described in greater detail. Purge valve 230 includes purge valve housing 232, purge valve body 234, and purge valve cap 236. Purge valve housing 232 has an aperture 231, which is configured to transmit pressure changes from inside the humidifier bottle. Purge valve body 234 includes a widened portion 235 and a plug 233. Plug 233 at least partially fits within aperture 231 when purge valve body 234 is in its resting position (as shown in FIG. 10A). Purge valve cap 236 may be fixedly attached to purge valve housing 232, or may fit on housing 232 with a snap-fit connection or the like. Purge valve body 234 fits and is slidable within housing 232. Body 234 is prevented from being withdrawn from housing 232 by virtue of widened portion 235. Widened portion.235 does not fit through the central opening in cap 236.

The purge valve shown in these figures is configured such that when a threshold level of pressure within main housing cap 215 is reached—typically due to kinked or otherwise obstructed tubing—sufficient pressure is transmitted through aperture 231 and into purge valve housing 232 to lift purge valve body 234 off of its resting position within the housing 232, as shown in FIG. 10B. Purge valve body 234 slides through the opening in purge valve cap 236 until the widened portion 235 of purge valve body 234, which does not fit through the opening in cap 236, hits the purge valve cap 236. When the gas released from the main housing cap 215 drops the pressure below the threshold level of pressure, the purge valve body 234 again drops to its position against the purge valve housing 232, with plug 233 positioned within aperture 231, as depicted in FIG. 10A.

In this manner, when the pressure within the device has exceeded an acceptable level, an audible “clicking” noise will be generated, thereby warning a patient or caregiver that there may be an obstruction in the tubing exiting the bottle from the gas outlet. The clicking noise may result from the widened portion 235 of the purge valve body 234 hitting against the purge valve cap 236 and/or from the bottom of the purge valve body 234 hitting against the purge valve housing 232 (or plug 233 hitting against the portion of housing 232 which defines aperture 231) as the pressure causes the valve to move up and down.

Because the valve body moves up and down while purging excess pressure from the housing, the purge valve device may also provide a visual warning to patients, which may be particularly important for patients that are hearing impaired. In other words, because the valve body is configured such that it can rise above the valve housing, and because the humidifier bottle is configured such that a user of the humidifier bottle can view the valve body rising above the valve housing, a visual warning of increased pressure within the humidifier bottle is provided. A portion of the valve body may also be brightly colored to further increase its visibility while positioned outside of the valve housing. In particular, in some embodiments the entire valve body, or at least a portion of the valve body which is visible when the valve body rises above the valve housing, is brightly colored.

Without further elaboration, it is believed that one skilled in the art can use the preceding description to utilize the invention to its fullest extent. The examples and embodiments disclosed herein are to be construed as merely illustrative and not a limitation of the scope of the present invention in any way. It will be apparent to those having skill in the art that changes may be made to the details of the above-described embodiments without departing from the underlying principles of the invention. In other words, various modifications and improvements of the embodiments specifically disclosed in the description above are within the scope of the appended claims. The scope of the invention is therefore defined by the following claims.

1. A humidifier bottle apparatus, comprising:
a main housing;
a fill shaft extending at an angle from the main housing;
a main housing cap connected with the main housing, wherein the main housing cap is substantially dome-shaped;
a gas inlet port connected with the main housing cap; and
a gas outlet port connected with the main housing cap, wherein the gas outlet port comprises a nozzle that is configured to swivel within the gas outlet port.

2. The humidifier bottle apparatus of claim 1, further comprising a fill shaft lid connectable with the fill shaft.

3. The humidifier bottle apparatus of claim 1, wherein the main housing cap is releasably connected with the main housing.

4. The humidifier bottle apparatus of claim 1, wherein the fill shaft comprises a fill shaft opening, and wherein the height of the main housing is greater than or equal to the height of the lowest point of the fill shaft defining the fill shaft opening.

5. The humidifier bottle apparatus of claim 1, further comprising a purge valve configured to provide an audible warning of increased pressure within the humidifier bottle.

6. The humidifier bottle apparatus of claim 5, wherein the purge valve comprises a valve body and a valve housing, wherein the valve body is positioned within and movable within the valve housing.

7. The humidifier bottle apparatus of claim 6, wherein the audible warning comprises a clicking noise generated when the valve body moves up and down within the valve housing.

8. The humidifier bottle apparatus of claim 7, wherein the valve body is configured such that it can rise above the valve housing, and wherein the humidifier bottle is configured such that a user of the humidifier bottle can view the valve body rising above the valve housing, thereby providing a visual warning of increased pressure within the humidifier bottle.

9. The humidifier bottle apparatus of claim 8, wherein at least a portion of the valve body which is visible when the valve body rises above the valve housing is brightly colored.

10. A humidifier bottle apparatus, comprising:
a main housing;
a fill shaft extending at an angle from the main housing;
a main housing cap connected with the main housing;
a purge valve configured to provide an audible warning of increased pressure within the humidifier bottle;
a gas inlet port connected with the main housing cap; and
a gas outlet port connected with the main housing cap.

11. The humidifier bottle apparatus of claim 10, wherein the purge valve is connected with the main housing cap.

12. The humidifier bottle apparatus of claim 10, wherein the main housing cap is releasably connected with the main housing.

13. The humidifier bottle apparatus of claim 10, wherein the gas outlet port comprises a nozzle that is configured to swivel within the gas outlet port.

14. The humidifier bottle apparatus of claim 10, wherein the main housing cap is substantially dome-shaped.

15. The humidifier bottle apparatus of claim 10, wherein the purge valve comprises a valve body and a valve housing, wherein the valve body is positioned within and movable within the valve housing.

16. The humidifier bottle apparatus of claim 15, wherein the audible warning comprises a clicking noise generated when the valve body moves up and down within the valve housing.

17. The humidifier bottle apparatus of claim 16, wherein the valve body is configured such that it can rise above the valve housing, and wherein the humidifier bottle is configured such that a user of the humidifier bottle can view the valve body rising above the valve housing, thereby providing a visual warning of increased pressure within the humidifier bottle.

18. The humidifier bottle apparatus of claim 17, wherein at least a portion of the valve body which is visible when the valve body rises above the valve housing is brightly colored.

19. The humidifier bottle apparatus of claim 10, wherein the fill shaft comprises a fill shaft opening, and wherein the height of the main housing is greater than or equal to the height of the lowest point of the fill shaft defining the fill shaft opening.

20. A humidifier bottle apparatus, comprising:
a main housing;
a fill shaft extending at an angle from the main housing;
a substantially dome-shaped main housing cap releasably connected with the main housing;
a purge valve connected with the main housing cap and configured to provide an audible warning of increased pressure within the humidifier bottle, wherein the purge valve comprises a valve body and a valve housing, wherein the valve body is positioned within and movable within the valve housing, wherein the audible warning comprises a clicking noise generated when the valve body moves up and down within the valve housing, and wherein the valve body is configured such that it can rise above the valve housing such that a user of the humidifier bottle can view the valve body rising above the valve housing, thereby providing a visual warning of increased pressure within the humidifier bottle;
a gas inlet port connected with the main housing cap; and
a gas outlet port connected with the main housing cap.

21. The humidifier bottle apparatus of claim 20, wherein the gas outlet port comprises a nozzle that is configured to swivel within the gas outlet port.
